(19) United States
(12) Patent Application Publication
(10) Pub. No.: US 2016/0106461 A1
(43) Pub. Date: Apr. 21, 2016

(54) SAFETY CANNULA

(71) Applicant: ROBERT EDWARD MORRIS, BIRMINGHAM, AL (US)

(72) Inventor: ROBERT EDWARD MORRIS, BIRMINGHAM, AL (US)

(21) Appl. No.: 14/982,294

(22) Filed: Dec. 29, 2015

Related U.S. Application Data
(63) Continuation of application No. PCT/US2014/044771, filed on Jun. 29, 2014.
Application No. PCT/US2014/044771 is a continuation of application No. 14/318,649, filed on Jun. 29, 2014, now abandoned.
(60) Provisional application No. 61/841,321, filed on Jun. 29, 2013.
(60) Provisional application No. 61/841,321, filed on Jun. 29, 2013.

(51) Int. Cl. A61B 17/34; A61B 17/02

(52) U.S. Cl. CPC A61B 17/3423; A61B 17/0218; A61B 17/0231; A61B 2017/349; A61B 2017/3486

(57) ABSTRACT

A cannula with a top section and tubular cylinder or sleeve designed to resist outward slippage during surgical manipulations, including but not limited to surgical manipulations of the eye. Means attached to the top section or the tubular cylinder or sleeve securely holds or fastens the cannula in place. Secure attaching is provided by threads, bladders, or expansile elements on the tubular cylinder or sleeve, or pincers extending from the top section, or combinations thereof.

This application is a continuation of PCT/US2014/44771, filed Jun. 29, 2014, which claims benefit of and priority to U.S. Provisional Application No. 61/841,321, filed Jun. 29, 2013, by Robert Edward Morris, and is entitled to those filing dates for priority. This application also is a continuation of U.S. patent application Ser. No. 14/318,649, filed Jun. 29, 2014, which claims benefit of and priority to U.S. Provisional Application No. 61/841,321, filed Jun. 29, 2013, by Robert Edward Morris, and is entitled to those filing dates for priority. The specifications, figures and complete disclosures of U.S. Provisional Application No. 61/841,321, U.S. patent application Ser. No. 14/318,649 and PCT/US2014/44771 are incorporated herein in their entireties by specific reference for all purposes.

### FIELD OF INVENTION

This invention relates to an improved cannula for use in surgical procedures, including, but not limited to, vitrectomy eye surgery.

### BACKGROUND OF THE INVENTION

Pars plana (closed) vitrectomy (referred to herein as “vitrectomy”) is a form of eye surgery in which vitreous gel is removed from the eye through tiny scleral incisions in the pars plana area of the eye. These incisions allow insertion of three devices into the vitreous cavity: (1) an infusion line, which keeps the eye inflated at desired pressure while vitreous gel is evacuated; (2) a suction/cutter probe to remove the vitreous gel by using selected vacuum levels while minimizing traction on the retinal tissue by frequent “guillotine” cutting at the aspiration port (typically 5000 cpm); and (3) a fiber optic probe to illuminate the vitreous cavity while viewing through the dilated pupil using a microscope.

From its invention in 1971 until approximately 2002, scleral incisions in vitrectomy (sclerotomies) were made by spear-shaped blades inserted radially. Infusion lines, fiber optics, and vitrectomy probes were inserted directly through these “bare sclera” incisions. Infusion lines were sutured to the sclera for assured security throughout the vitrectomy operation, and confirmed to extend fully into the vitreous cavity by direct viewing through the pupil prior to starting infusion.

Since approximately 2002, vitrectomy surgery has with increasing frequency been performed through metal “cannulas” inserted through both the sclera and overlying conjunctival membrane. A “trocar” spear-shaped blade is used to produce the sclerotomy and a hollow cannula riding on the trocar blade shaft is inserted into the sclera as the trocar is inserted through the sclera. The cannula remains after the trocar is removed and throughout the vitrectomy surgery. The infusion line is placed into one of three cannulas, and is visually confirmed to be completely into the vitreous cavity, penetrating the full thickness of the eye wall. The eye wall consists of the tough outer, white sclera; the vascular choroid layer; and the neurosensory layer, posteriorly called the retina. The fiber optic probe and vitrectomy probe are placed directly through the remaining two cannulas. An example of a trochar and cannula are disclosed in Spaide, U.S. Pat. No. 8,287,560, which is incorporated herein by specific reference for all purposes.

The advantages of cannulating scleral incisions are that it allows vitrectomy without incision and dissection of the overlying conjunctiva, and production of scleral wounds that are typically self-sealing upon cannula removal at the completion of the vitrectomy surgery. This eliminates the need for suturing. Thus, the operation is shorter, both on entrance and in closing. Even more importantly, the eye surface is left with minimal evidence that vitrectomy has been performed, with dramatic improvements in patient comfort and cosmesis, and reduced surface scarring to inhibit future eye operations.

However, there are significant disadvantages with the prior art. Cannulas are not secured to the sclera by suturing. Current vitrectomy cannulas consist of hollow, cylinder shapes made from metal, so as to be easily inserted and removed, with least possible friction for both insertion and removal, and also for low friction instrument manipulation through the cannulas. But the advantages of low friction are disadvantages for cannula position security, resulting in a propensity toward outward slippage. Consequently, it is common to detect cannula outward slippage during the course of vitrectomy, as the eye is manipulated. When detected, the slippage can be remedied by simply pushing the cannula back into the scleral wound. Outward slipping of the fiber optic and vitrectomy cannulas is typically only an annoyance. But if the infusion line cannula slips out undetected (the operating room is kept dark, making the external eye invisible to the surgeon during most of the operation), the infusion of either liquid or air can be accidentally directed between the scleral wall and the underlying choroid vascular layer, producing a choroidal detachment. If liquid is being infused, the choroidal detachment may necessitate external drainage or may be so extensive as to require cessation of surgery, and it can occasionally injure the eye permanently. If air is being infused, cannula slippage can rarely cause air under pressure to be infused between the sclera and choroid, tearing the vortex vein outflow, causing air embolization into the heart. This can be fatal if not promptly detected. As well, the infusion line cannula could slip completely out of the eye wall while the surgical instruments remain in the eye, causing ocular collapse and consequent intraocular damage.

Accordingly, what is needed is an improved cannula for use in various forms of surgical procedures, particularly for use with the infusion line, that overcomes these disadvantages.

### SUMMARY OF INVENTION

In various exemplary embodiments, the present invention comprises a cannula designed to resist outward slippage during surgical manipulations of the eye or other tissue. In several embodiments, the cannula comprises means for securely holding or fastening the cannula in place to the eye wall or other tissue. While the embodiments discussed below are in the in context of eye surgery, cannulae in accordance with the present invention can be used in surgeries in other parts of the body.

In one exemplary embodiment, the improved cannula comprises a distal end that is inserted into the sclera, eye wall, or other tissue, and a proximal end that remains on the exterior of the eye or tissue after insertion. The proximal end comprises a top section into which the trochar or other instrument can be inserted. A tubular cylinder or sleeve, generally with a diameter less than that of the top section, extends from the distal side of the top section, defining an opening that extends through the top section and the length of the cannula.

In some embodiments, the outside of the tubular sleeve comprises a helical or screw-like thread. The threaded cannula prevents typical outward slippage of the cannula caused by modest linear forces when the cannula is pulled on by eye movement or manipulation through surgery. Easy insertion and removal is accomplished by rotation of the cannula in the appropriate clockwise or counterclockwise direction.

In additional exemplary embodiments, the cannula comprises one or more inner bladders located on the tubular sleeve. The inner bladders are in fluid communication with an inflation port or outer bladder located on the top section. The inner bladders are uninflated during insertion of the cannula, and once in place, the surgeon injects fluid or gas into the inflation port or outer bladder, thereby cause temporary inflation of the inner bladders. The inflated inner-bladders hold the cannula in place against outward slippage. In one embodiment, the outer bladder has a thicker wall of greater strength than the inner bladders, thereby resisting excessive inflation. At the conclusion of the surgery, the surgeon can release the inflation port or cut the outer bladder or aspirate fluid to cause deflation of the inner bladders and allow for easy removal of the cannula.

In further exemplary embodiments, the cannula comprises one or more expansile elements located on the tubular sleeve. The expansile elements are flattened or compressed against the tubular sleeve when inserted, but after insertion, expand outward, thereby making the cannula self-retaining and resistant to removal due to typical inadvertent outward forces that occur during eye surgery manipulations. At the conclusion of the surgery, removal requires an substantial outward linear pulling force.

The expansile element is designed to be relatively easy to insert but harder to remove. Compression of the expansile element during insertion may be facilitated by a relatively small, acute angle of the expansile element in relation to the trocar shaft at the end of the expansile element near the trocar entry tip. There is a greater angle of the expansile element relative to the trocar shaft at the upper end of the expansile element. Compression of the expansile element may be aided by linear fenestrations parallel to the long axis. The expansile element may be made of thin, flexible material, with low friction to entry and exit relative to the scleral tissue.

Any number of expansile elements, evenly or irregularly spaced, may be used. In one embodiment, both ends of the expansile element are fixed to the tubular sleeve with the expansile element expanding at or near the middle, while in other embodiments one end of the expansile element is fixed to the tubular sleeve, while the other end may be affixed to ring encompassing the tubular sleeve and able to slide up and down thereon.

In several embodiments, the expansile elements alone form a substantial section of the tubular sleeve itself, with open space therebetween. The tubular sleeve comprises an upper section extending through the sclera or tissue, a center section comprising the expansile elements (which can be any number of elements), and a lower ring section serving to attach the lower ends of the expansile elements. In one embodiment, a solid tube or sleeve (e.g., an infusion cannula) at the end of an infusion line or instrument can be inserted into and attached to the cannula described herein (e.g., by threads, or a screw or clip) to provide support and a solid sleeve throughout the entire length of cannula.

In another embodiment, one or more pincers with sharp points extend from the top section and engage the outer surface of the sclera. Any number of pincers may be used, and they may be evenly or irregularly spaced. The pincers may extend from the top at an angle, so that the points can be pushed into the outer surface of the sclera with a twisting motion. Removal is accomplished by reversing the twisting motion, thus making the cannula resistant to removal due to typical inadvertent outward linear forces that occur during eye surgery manipulations.

In a further embodiment, the present invention comprises a means for connecting an infusion line to the top section of a cannula. Threads on the end of the infusion line engage matching threads on the interior of the top section. Single or multiple threads may be provided.

While the above embodiments have been described independently, combinations thereof may, of course, be used.

### BRIEF DESCRIPTION OF THE DRAWINGS

FIGS. 1-3 shows views of a cannula with threads in accordance with an embodiment of the present invention.

FIGS. 4-7 shows views of a cannula with bladders in accordance with an embodiment of the present invention.

FIGS. 8-13 shows views of a cannula with expansile elements in accordance with an embodiment of the present invention.

FIGS. 14-15 shows views of a cannula with pincers in accordance with an embodiment of the present invention.

FIGS. 16-17 show views of a cannula and infusion line with engaging threads.

### DETAILED DESCRIPTION OF EXEMPLARY EMBODIMENTS

In various exemplary embodiments, the present invention comprises a cannula designed to resist outward slippage during surgical manipulations of the eye or other tissue. In several embodiments, the cannula comprises means for securely holding or fastening the cannula in place to the eye wall or other tissue. While the embodiments discussed below are in the in context of eye surgery, cannulae in accordance with the present invention can be used in surgeries in other parts of the body.

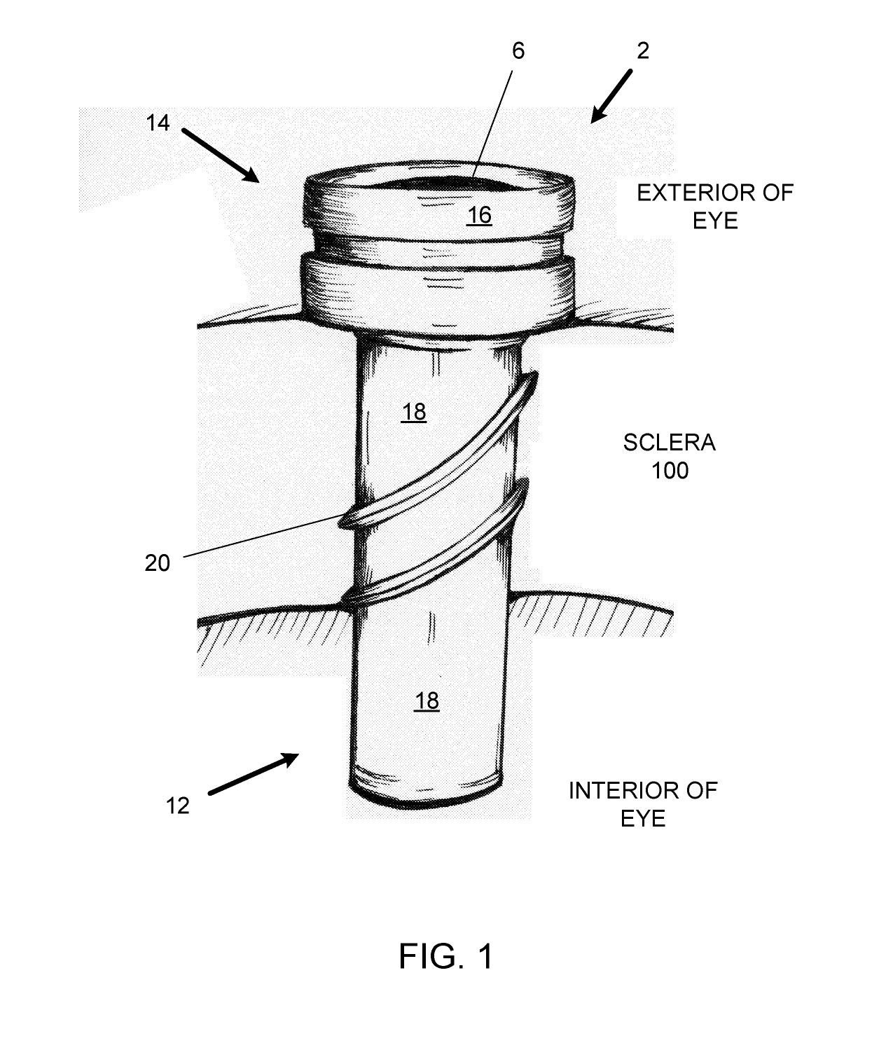

In one exemplary embodiment, as seen in FIG. 1, the improved cannula 2 comprises a distal end 12 that is inserted into the sclera, eye wall, or other tissue 100, and a proximal end 14 that remains on the exterior of the eye or tissue after insertion. The proximal end 14 comprises a top section 16 into which the trochar (generally comprising a trochar blade 110 at the end of a trochar shaft 112) or other instrument or line can be inserted. A tubular cylinder or sleeve 18, generally with a diameter less than that of the top section, extends from the distal side of the top section, defining an opening 6 that extends through the top section and the length of the cannula.

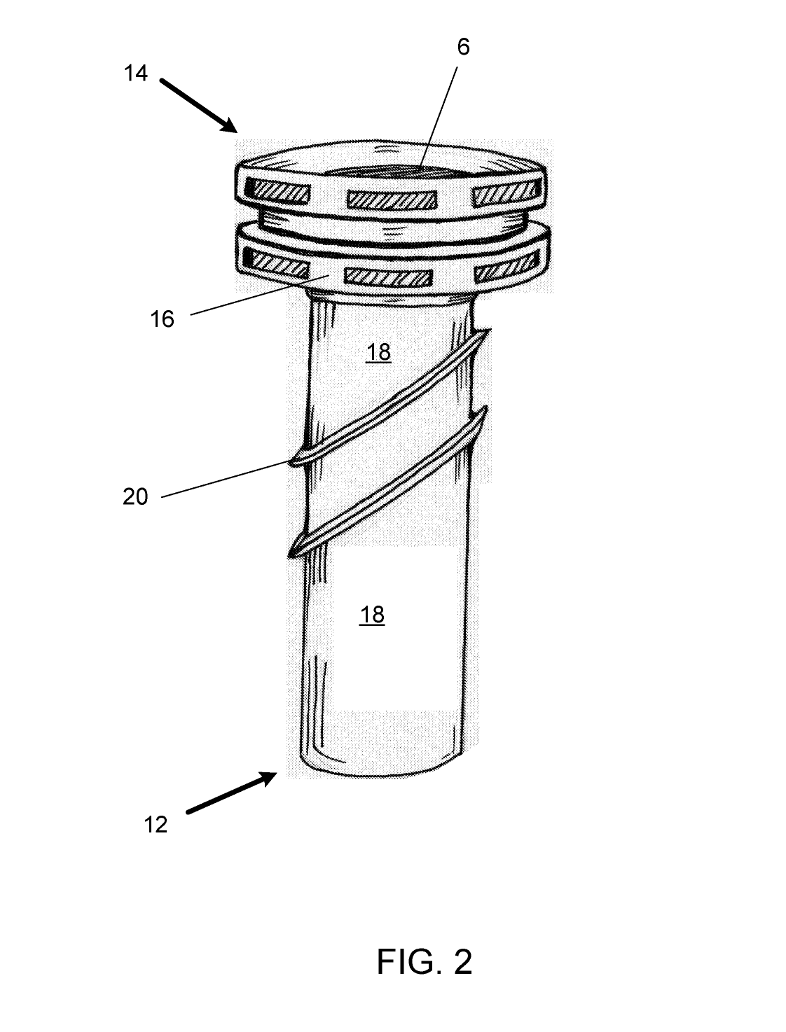

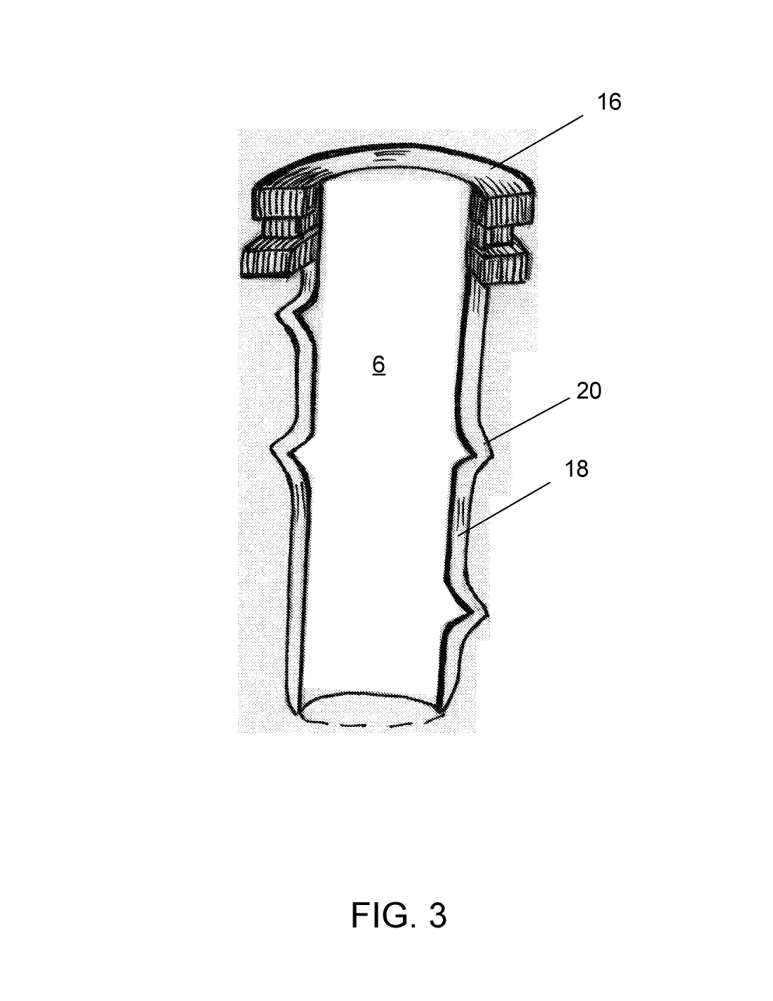

In the embodiment shown in FIGS. 1-3, the exterior of the tubular sleeve 18 comprises a helical or screw-like thread 20. The threaded cannula prevents typical outward slippage of the cannula 2 caused by modest linear forces when the cannula is pulled on by eye movement or manipulation through surgery. Easy insertion and removal is accomplished by rotation of the cannula in the appropriate clockwise or counterclockwise direction.

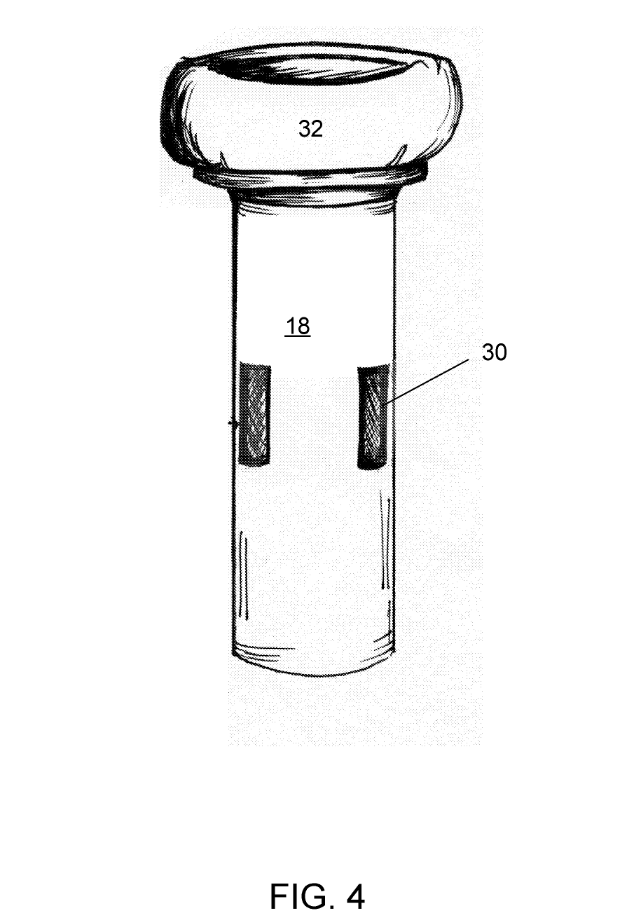

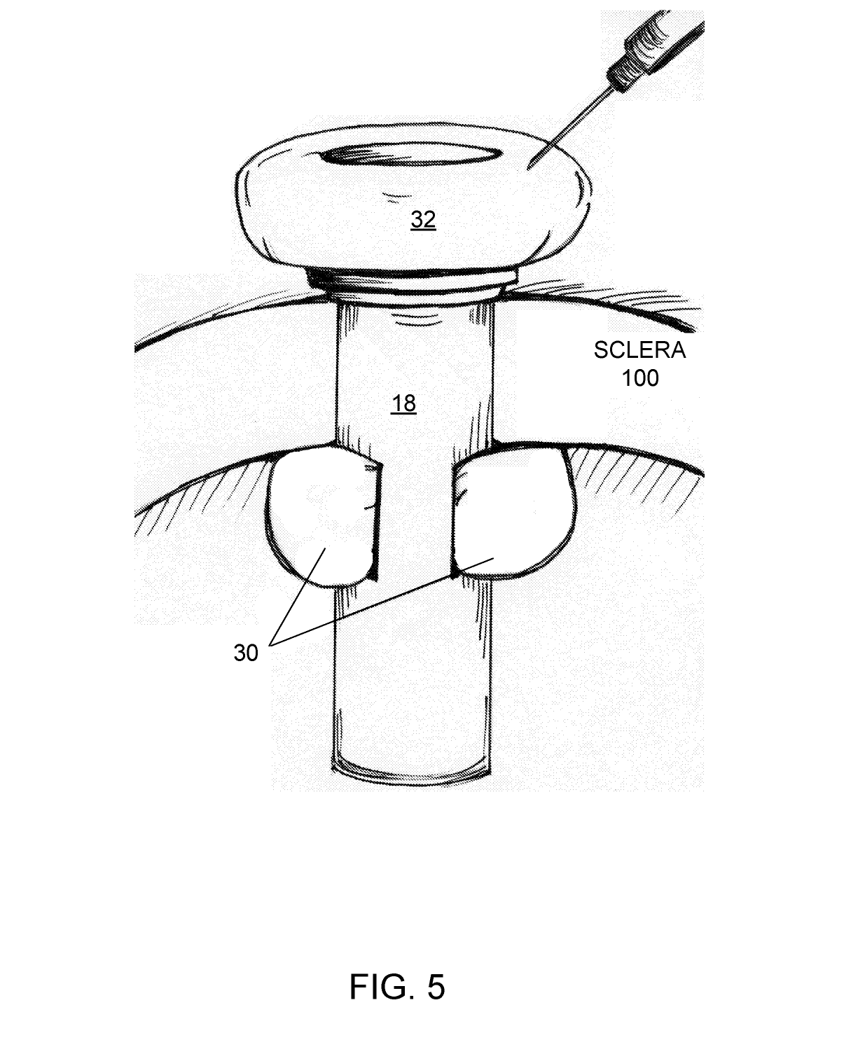

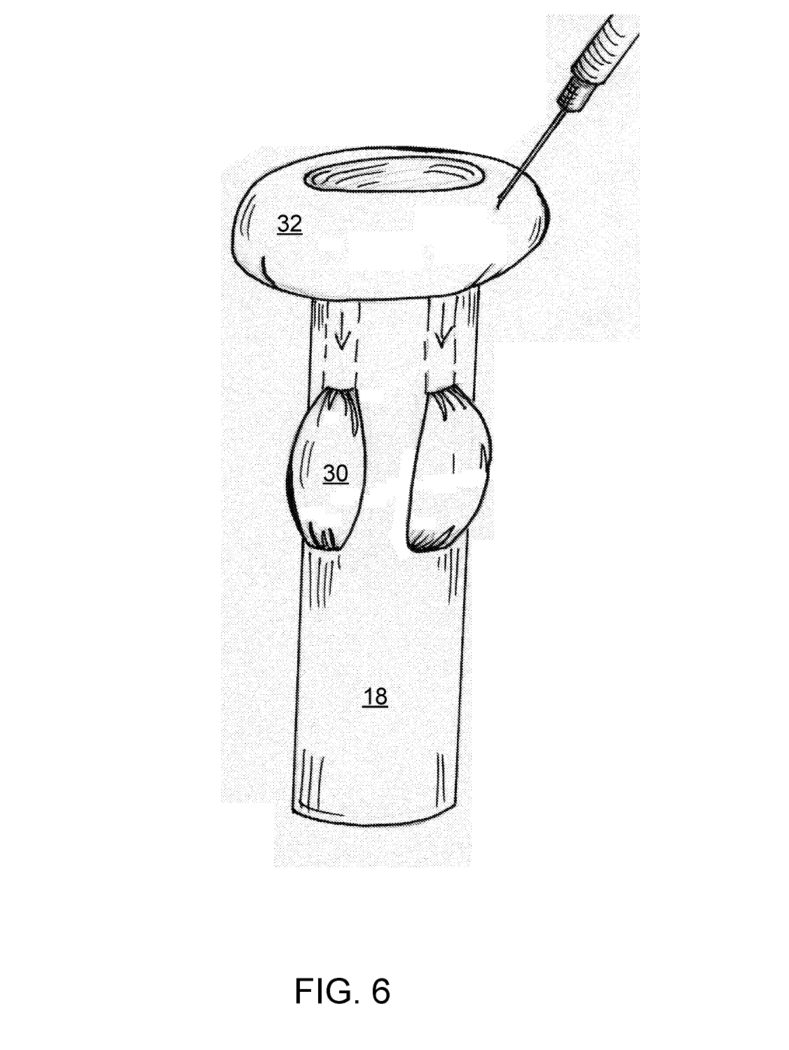

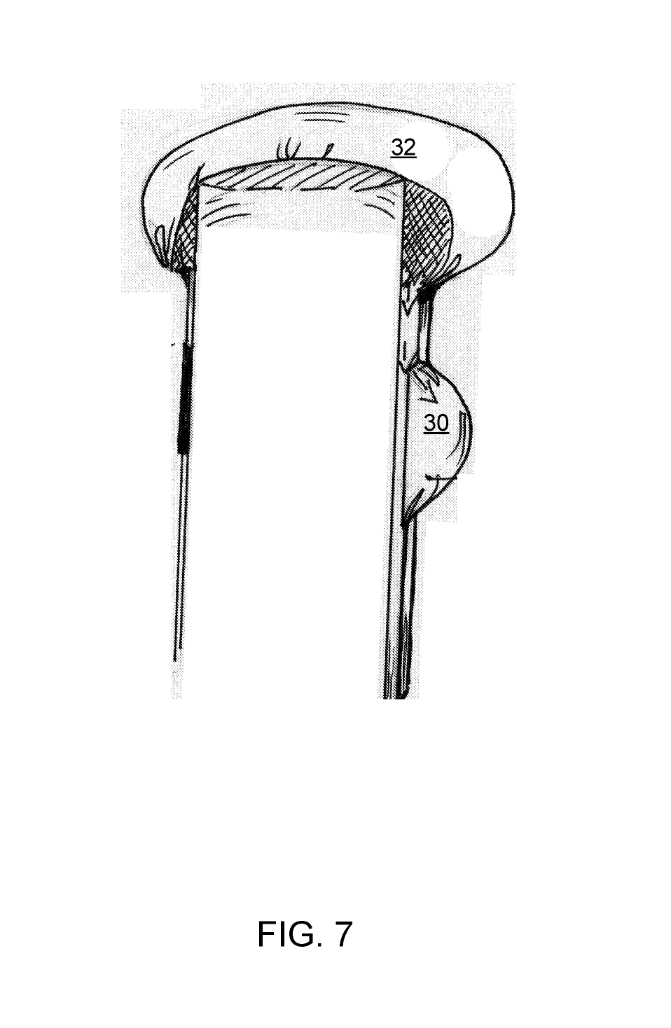

In the embodiment shown in FIGS. 4-7, the cannula comprises one or more inner bladders 30 located on the tubular sleeve 18. The inner bladders 30 are in fluid communication with an inflation port or outer bladder 32 located on the top section 16. The inner bladders are uninflated during insertion of the cannula, and once in place, the surgeon injects fluid or gas into the inflation port or outer bladder, thereby cause temporary inflation of the inner bladders. The inflated inner-bladders 30 hold the cannula in place against outward slippage. In one embodiment, the outer bladder 32 has a thicker wall of greater strength than the inner bladders, thereby resisting excessive inflation. At the conclusion of the surgery, the surgeon can release the inflation port or cut the outer bladder or aspirate fluid to cause deflation of the inner bladders and allow for easy removal of the cannula.

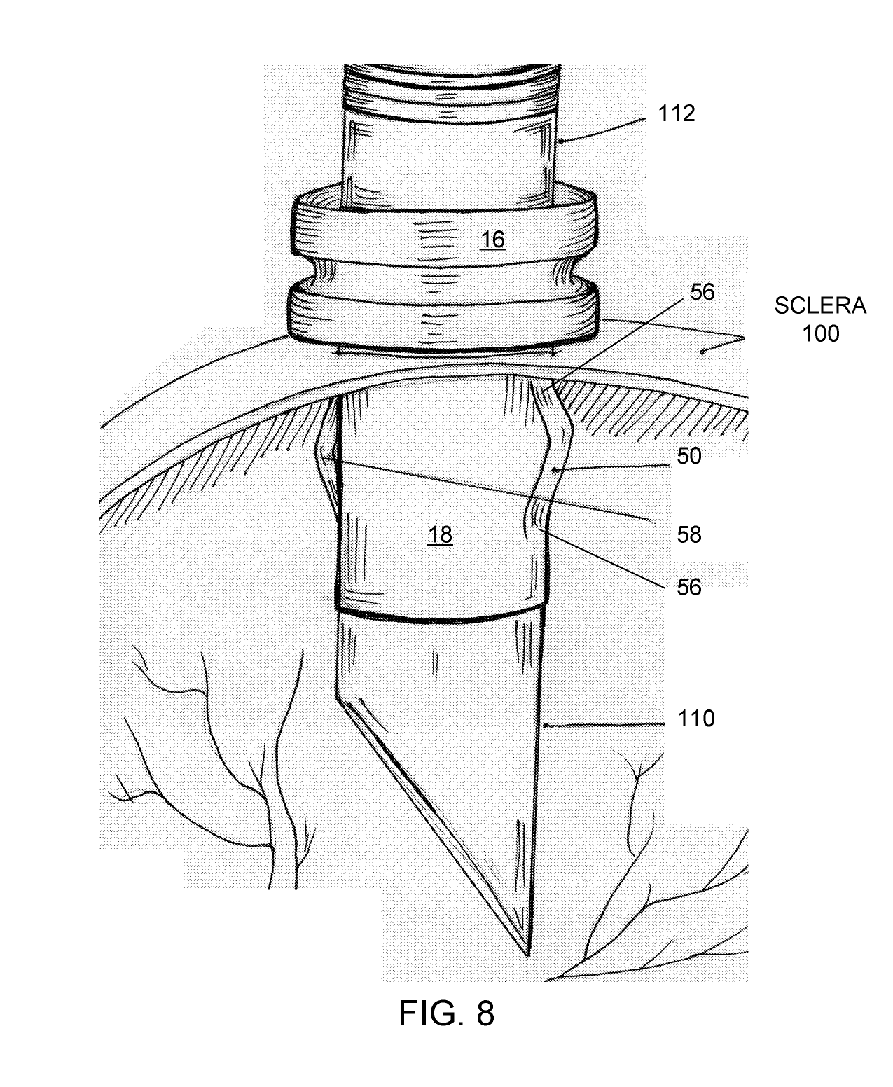

In the embodiment shown in FIGS. 8-13, the cannula comprises one or more expansile elements 50 located on or comprising part of the tubular sleeve 18. The expansile elements 50 are flattened or compressed against the tubular sleeve 18 when inserted, but after insertion, expand outward, thereby making the cannula self-retaining and resistant to removal due to typical inadvertent outward forces that occur during eye surgery manipulations. At the conclusion of the surgery, removal requires an substantial outward linear pulling force.

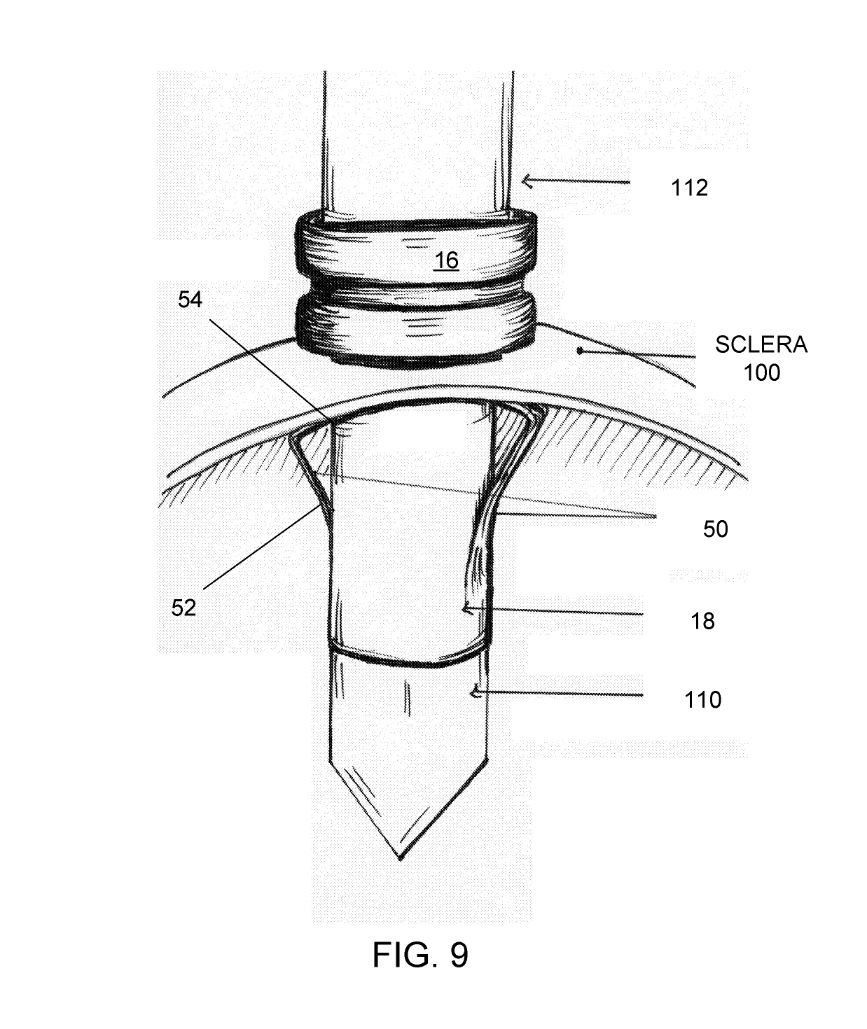

The expansile element is designed to be relatively easy to insert but harder to remove. As seen in FIG. 9, compression of the expansile element during insertion may be facilitated by a relatively small, acute angle 52 of the expansile element in relation to the trocar shaft at the end of the expansile element near the trocar entry tip. There is a greater angle 54 of the expansile element relative to the trocar shaft at the upper end of the expansile element. Compression of the expansile element may be aided by linear fenestrations parallel to the long axis. The expansile element may be made of thin, flexible material, with low friction to entry and exit relative to the scleral tissue. After insertion, the expansile element returns to its normal position (i.e., expanded position).

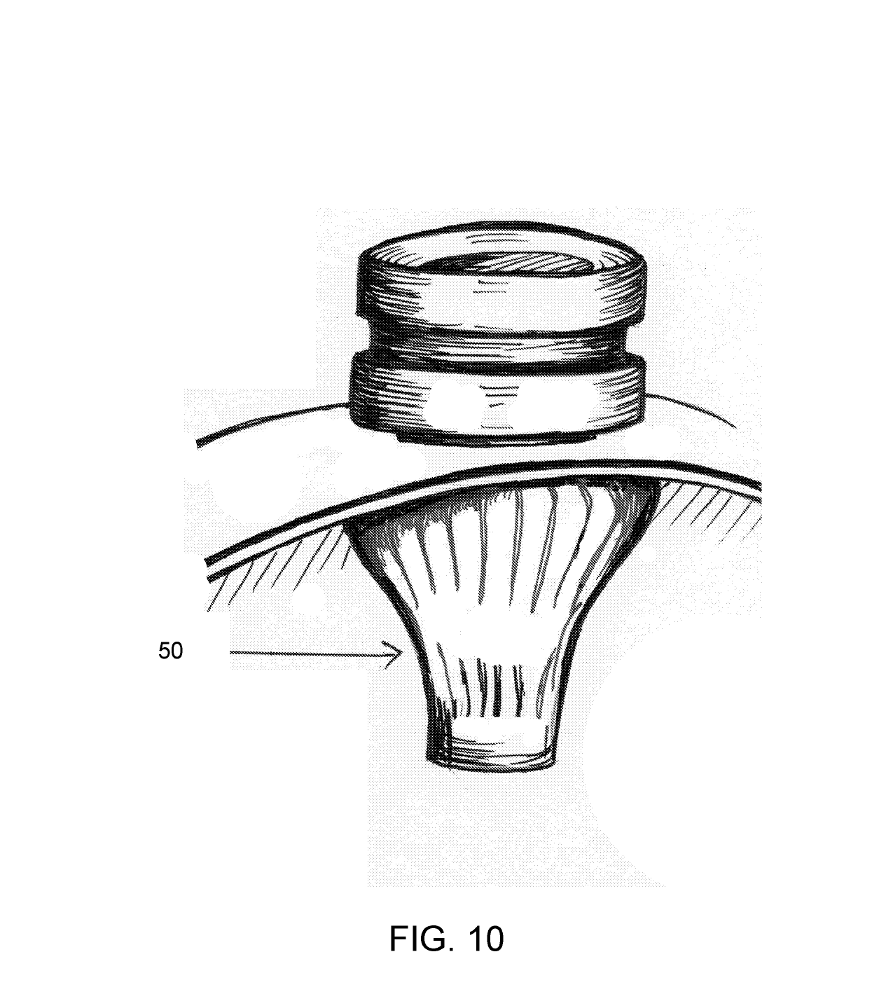

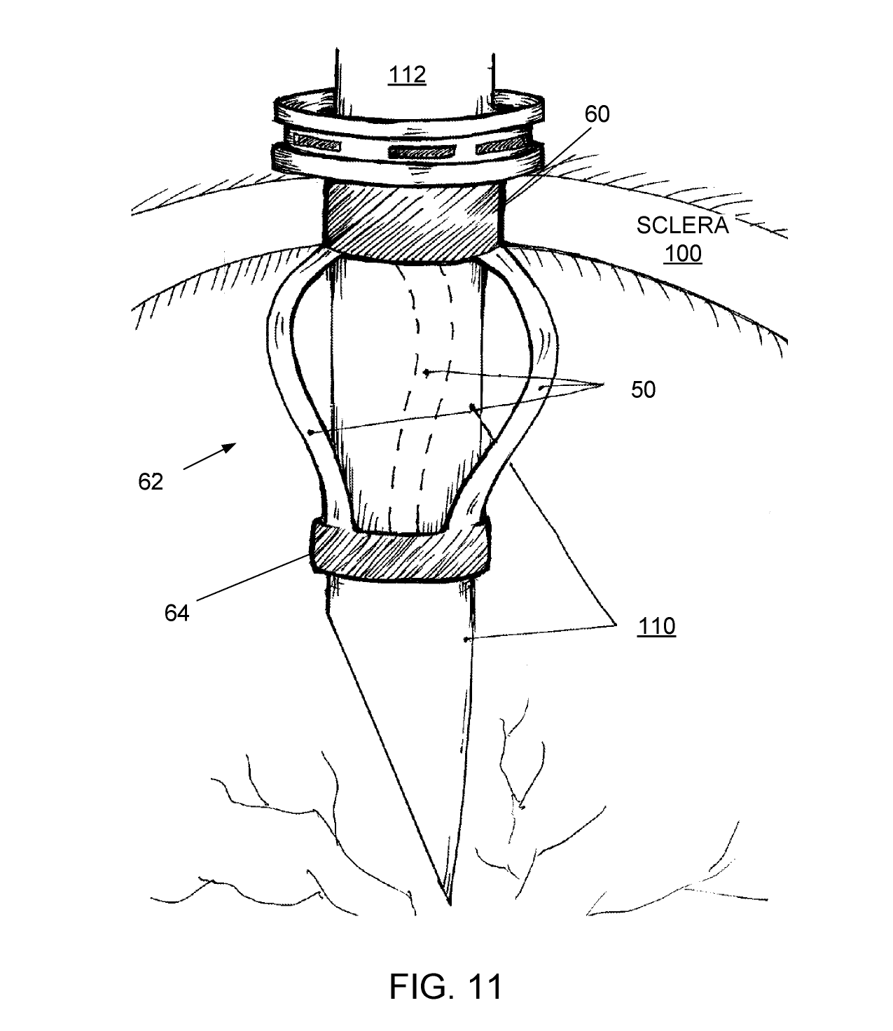

Any number of expansile elements, evenly or irregularly spaced, may be used. FIG. 9 shows an exemplary embodiment with two expansile elements 50 on opposite sides, while FIG. 10 shows multiple elements 50 (in essence, a 360-degree symmetric expansile). Three or four expansile elements 50, evenly spaced around the circumference of the tubular sleeve, also may be used, as seen in FIG. 11. In one embodiment, as seen in FIG. 8, both ends 56 of the expansile element are fixed to the tubular sleeve with the expansile element expanding at the middle 58, while in other embodiments one end of the expansile element is fixed to the tubular sleeve, while the other end may be affixed to a ring encompassing the tubular sleeve and able to slide up and down thereon.

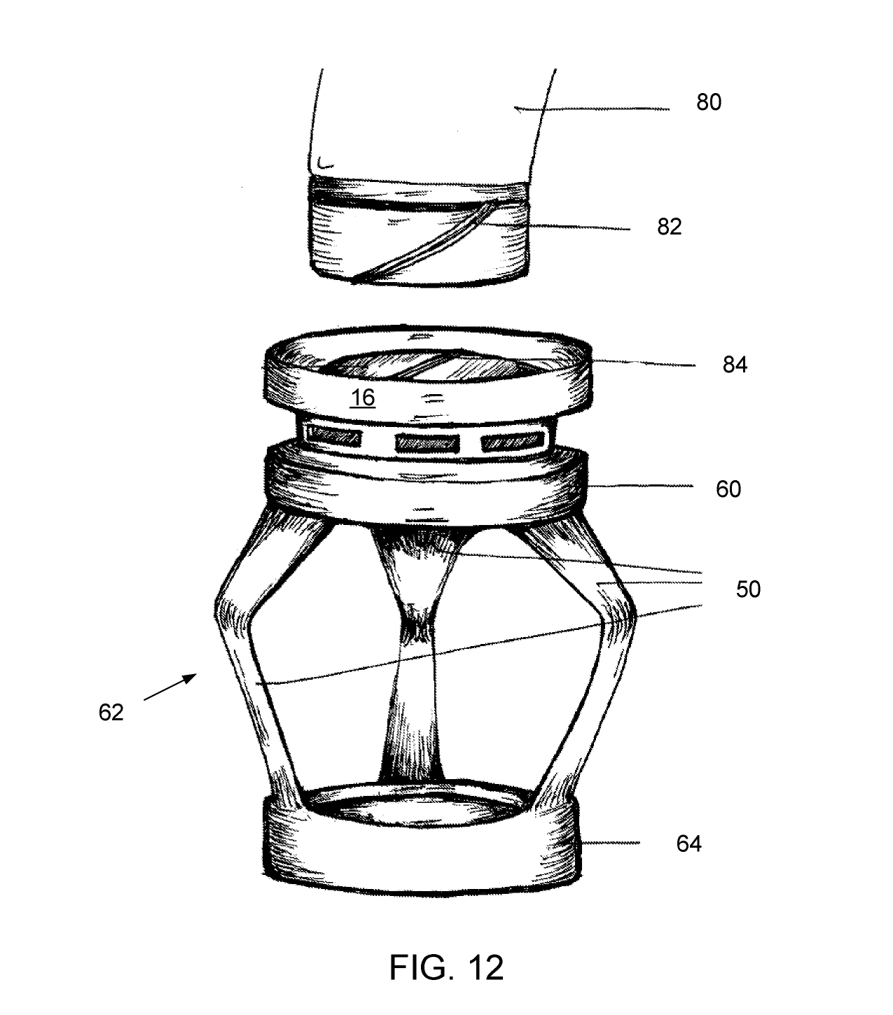

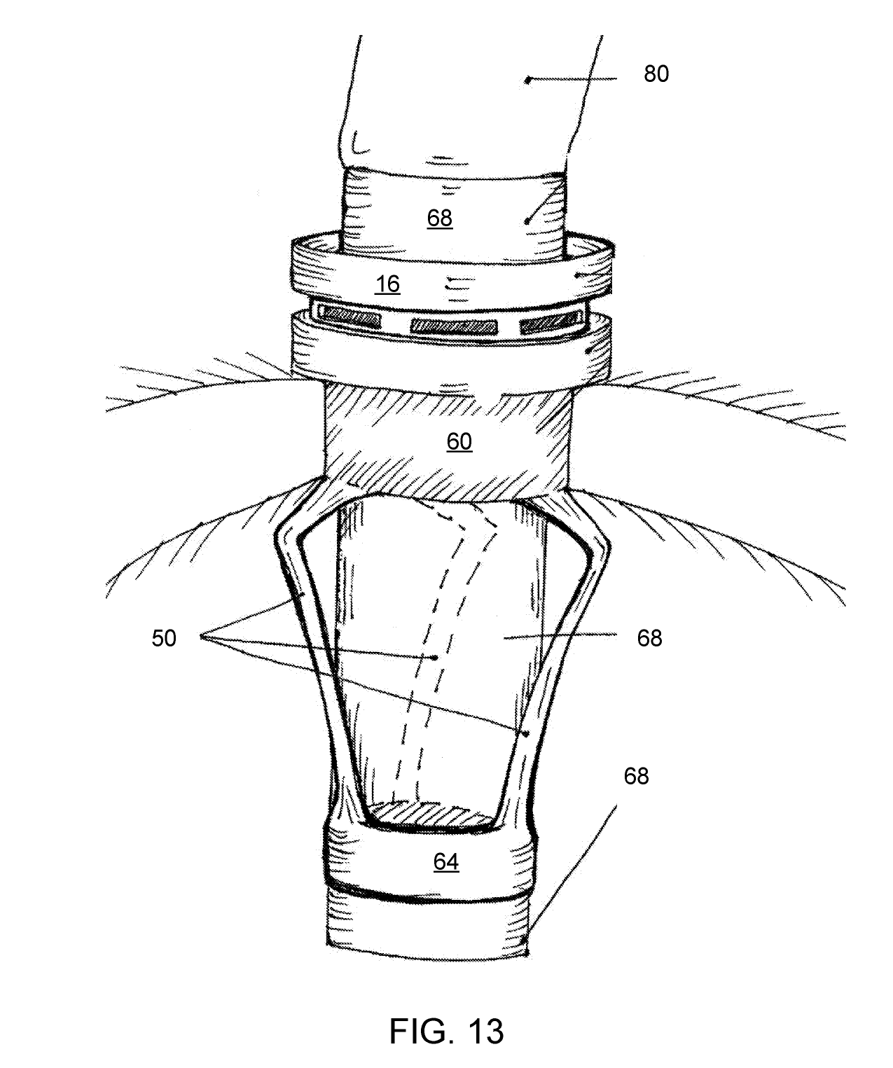

As seen in FIGS. 11 and 12, in several embodiments the expansile elements 50 alone form a substantial section of the tubular sleeve itself, with open space therebetween. In the embodiment shown, the tubular sleeve comprises an upper section 60 extending through the sclera or tissue, a center section 62 comprising the expansile elements (which can be any number of elements), and a lower ring section 64 serving to attach the lower ends of the expansile elements. In one embodiment, as seen in FIG. 13, a solid tube or sleeve 68 (e.g., an infusion cannula) at the end of an infusion line 80 or instrument can be inserted into and attached to the cannula described herein (e.g., by threads, or a screw or clip) to provide support and a solid sleeve throughout the entire length of cannula.

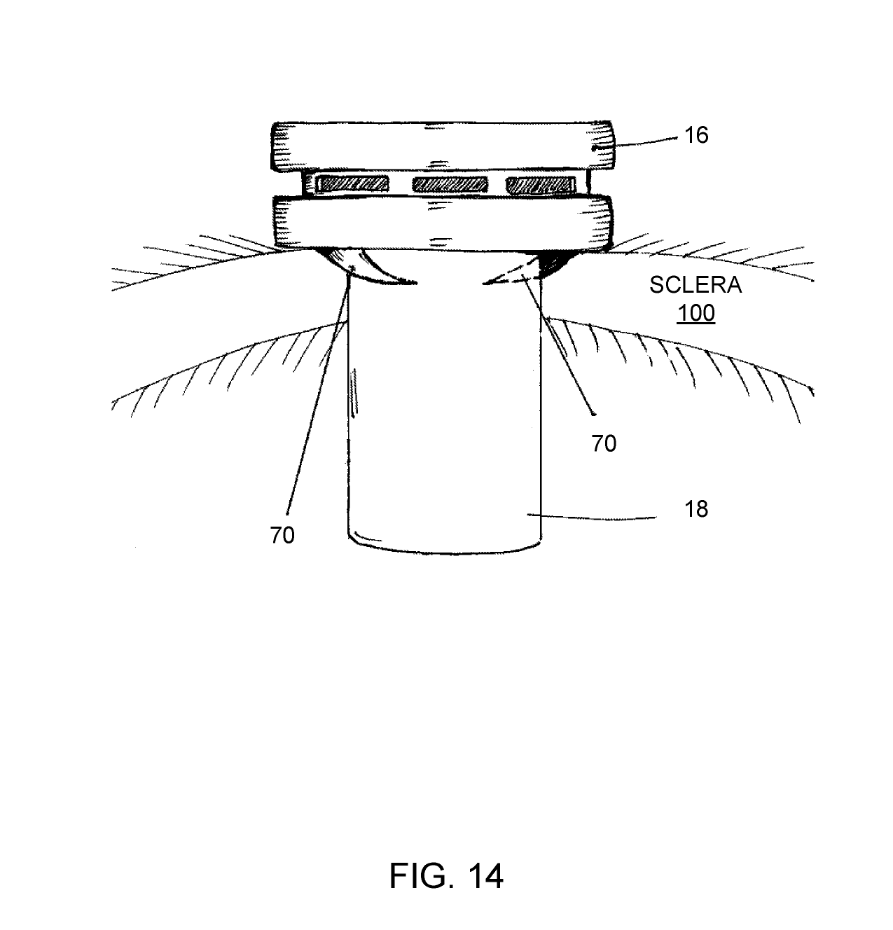

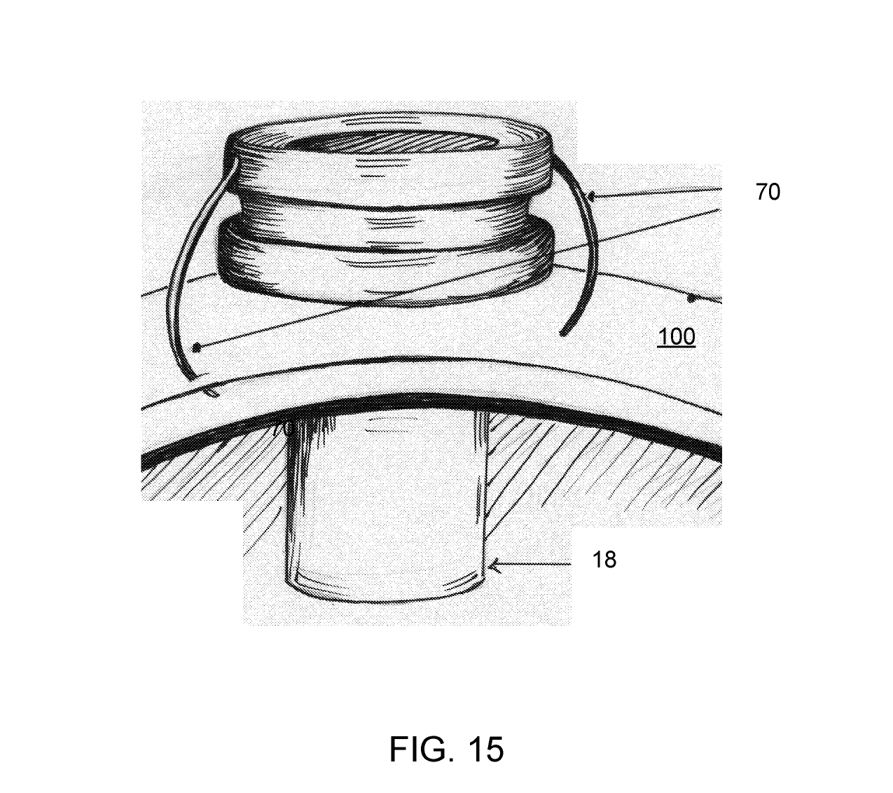

In the embodiment shown in FIGS. 14-15, one or more pincers 70 with sharp points extend from the top section and engage the outer surface of the sclera. Any number of pincers may be used, and they may be evenly or irregularly spaced. In the embodiment shown in FIG. 12, the pincers extend from the top at an angle, so that the points can be pushed into the outer surface of the sclera with a twisting motion. Removal is accomplished by reversing the twisting motion, thus making the cannula resistant to removal due to typical inadvertent outward linear forces that occur during eye surgery manipulations.

In the embodiment shown in FIG. 14, two pincers 70 extend from the underside of the top section. In FIG. 15, two pincers 70 extend from the sides of the top sections. The pincers may be flexible enough to be pulled outward and released for insertion, thereby creating tension in the pincers sufficient to resist removal of the cannula.

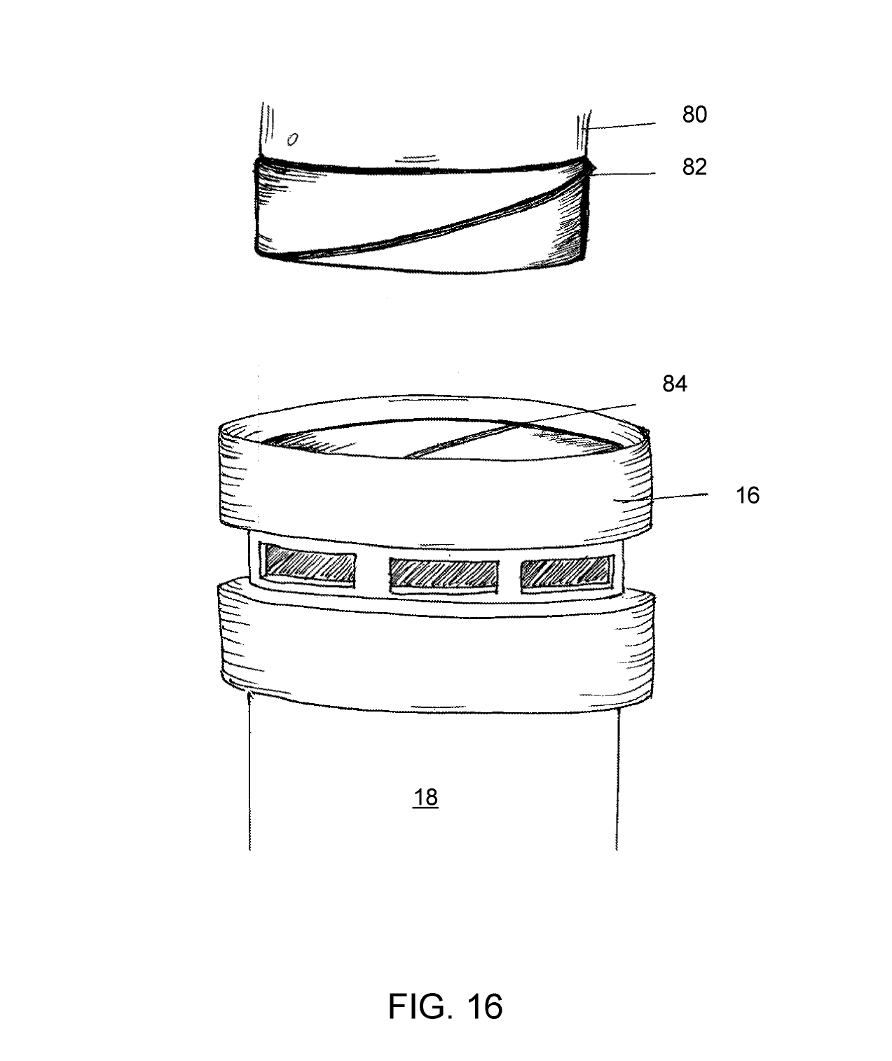

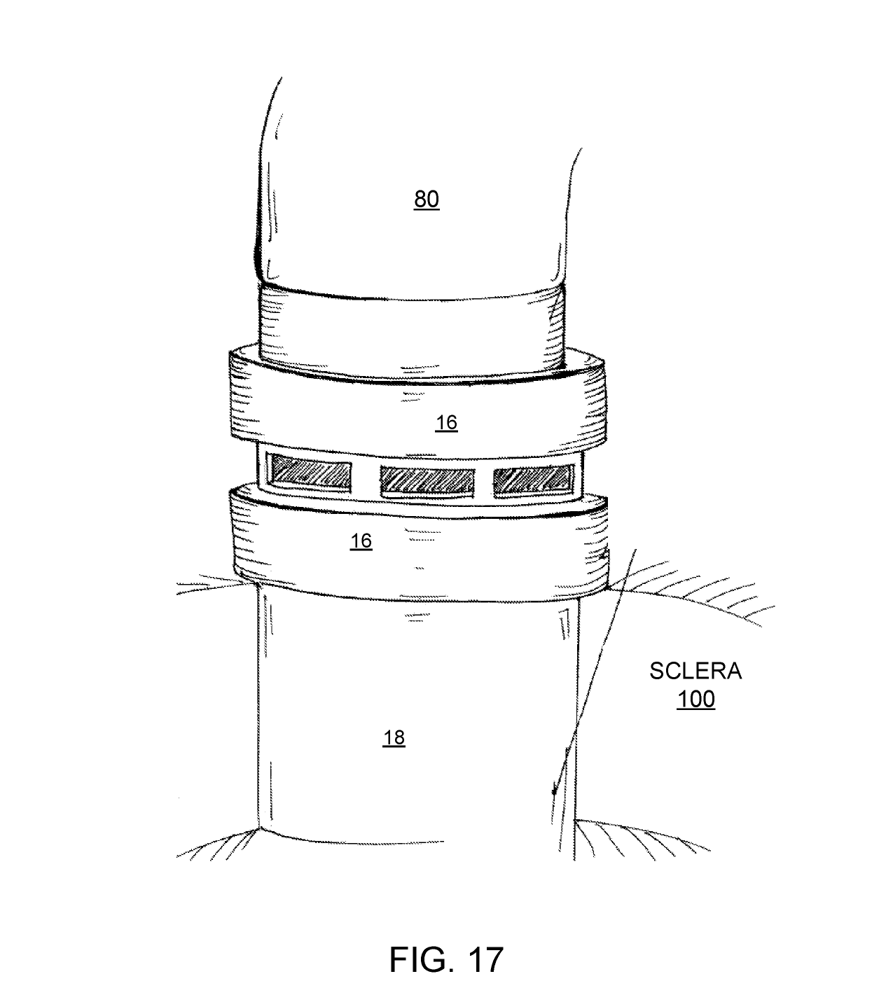

FIGS. 16 and 17 shows an example of a means for connecting an infusion line 80 to the top section of a cannula. Threads 82 on the end of the infusion line 80 engage matching threads 84 on the interior of the top section. Single or multiple threads may be provided.

While the above embodiments have been shown independently, combinations of the elements described therein may, of course, be used.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A device, comprising:
a cannula with a top section connected to a tubular cylinder or sleeve with an exterior and an interior, with an opening extending therethrough; and
means for securing the cannula during use.

2. The device of claim 1, the securing means comprising one or more threads on the exterior of said tubular cylinder or sleeve.

3. The device of claim 1, the securing means comprising one or more inner bladders positioned along the exterior of said tubular cylinder or sleeve.

4. The device of claim 3, further comprising means for inflating and deflating said one or more inner bladders.

5. The device of claim 4, wherein said inflating and deflating means comprising a port or outer bladder placed on or in said top section, said port or outer bladder in fluid communication with said one or more inner bladders.

6. The device of claim 1, the securing means comprising one or more expansile elements on the tubular cylinder or sleeve.

7. The device of claim 6, each expansile element comprising an upper end and a lower end.

8. The device of claim 7, wherein the upper end and lower end are fixedly attached to the exterior of the tubular cylinder or sleeve.

9. The device of claim 8, wherein the lower end is attached at an angle lesser than the angle at which the upper end is attached.

10. The device of claim 6, wherein there are three expansile elements.

11. The device of claim 6, wherein the expansile elements extend for the entire circumference of the tubular cylinder or sleeve.

12. The device of claim 6, wherein the expansile elements comprise a portion of the tubular cylinder or sleeve, with openings between adjacent expansile elements.

13. The device of claim 1, the securing means comprising one or more pincers extending from the top section.

14. The device of claim 13, wherein the pincers extend from an underside of the top section.

15. The device of claim 13, wherein the pincers extend from a side of the top section.

16. The device of claim 13, wherein there are two pincers.

17. The device of claim 13, wherein the pincers extend at an angle from the top section.

18. The device of claim 1, further comprising threads on the interior of the top section.

19. The device of claim 1, wherein the cannula is adapted to receive an air infusion line.

20. The device of claim 12, further comprising an insertion cylinder or sleeve, slidingly inserted behind the expansive elements.
